(19) United States
(12) Patent Application Publication
(10) Pub. No.: US 2021/0121252 A1
(43) Pub. Date: Apr. 29, 2021

(54) ROBOTIC INSTRUMENT FOR BONE REMOVAL

(71) Applicant: Eindhoven Medical Robotics B.V., Eindhoven (NL)

(72) Inventor: Jordan Bos, Kerkdriel (NL)

(73) Assignee: Eindhoven Medical Robotics B.V., Eindhoven (NL)

(21) Appl. No.: 17/046,688

(22) PCT Filed: Apr. 11, 2019
(86) PCT No.: PCT/NL2019/050217

§ 371 Date: Oct. 9, 2020

Related U.S. Application Data
(60) Provisional application No. 62/656,504, filed on Apr. 12, 2018.

(51) Int. Cl. A61B 34/32; A61B 17/16; A61B 90/14; A61B 90/00; A61B 6/03; A61B 34/10; A61B 6/00

(52) U.S. Cl. CPC A61B 34/32; A61B 17/1695; A61B 90/14; A61B 90/39; A61B 6/032; A61B 2017/00212; A61B 6/505; A61B 2034/105; A61B 2034/107; A61B 2090/3983; A61B 34/10

(57) ABSTRACT

Robot 1 for bone removal from the skull 2 of a patient which robot 1 comprises a base 3 connected to a robotic arm 4 comprising a series of joints 5 to 11, where the first joint 5 of the series is connected to the base 3 and the last joint 11 of the series is connected to a surgical instrument 12, so that the series of joints 5 to 11 provide degrees of freedom on different axes to the surgical instrument 12, which robot 1 is provided with a headrest 13 for the skull 2, where the headrest 13 is directly fixated to or is integrated in the base 3 of the robot, next to the first joint 5 of the series 5 to 11.

Robot for bone removal from the skull of a patient which robot comprises a base connected to a robotic arm comprising a series of joints, where the first joint of the series is connected to the base and the last joint of the series is connected to a surgical instrument, so that the series of joints provide degrees of freedom on different axes to the surgical instrument, which robot is provided with a headrest for the skull.

Such an instrument is known from Weber et al., Sci. Robot. 2, eaal4916 (2017) 15 Mar. 2017, where a drilling tool for bone removal is fixed to a robotic arm with a series of joints. The base of the robot is via an attachment fixed to an operating table. The headrest is mounted via a rail mechanism to the operating table. A problem of the known instrument is that it is difficult to maintain accuracy during bone removal.

According to the invention the headrest is directly fixated to or is integrated in the base of the robot, next to the first joint of the series. This means that the force loop between the bone removal instrument and the area of the skull where bone needs to be removed is short. In the prior art the manipulator length from base to a tool tip is 700 mm. The large forces when removing bone and the relatively low stiffness caused by the manipulator length of 700 mm give problems with accuracy. As a result of the short force loop the instrument of the invention offers a much higher stiffness and accuracy. The headrest can be part of the base of the robot or comprises an intermediate component that is fixed to the base of the robot.

Preferably the series of joints comprise revolute joints that are conceptually orthogonal with respect to each-other, where revolute joints have a distance between the joints that is slightly larger than a maximum diameter of the joints and where the last joint is a prismatic unit connected to the surgical instrument. Conceptually orthogonal means that the angle between the joints can vary from 80 to 100 degrees, with a preference for 90 degrees. This means that forces from the surgical instrument to the base can be transferred in a path as linear as possible. The revolute joints according to the invention have a minimal distance between the joints and a maximal diameter or dimension of the joints for increased bending stiffness and reduced inertia. In practice this means that the distance between the joints is slightly larger than a maximal diameter of the joint. Slightly larger means that the diameter (dimension transverse to the distance between the joints) is between 3 and 8% smaller than the distance. In a practical example of the robot the distance is 75 mm and the diameter 71 mm, i.e. around 5% less than the distance. The last joint is a prismatic unit, i.e. a linear moving unit, connected to the instrument. The distance and the diameter of the joints are determined by the space the robot is allowed to occupy, the reach required for the surgical instrument and the required stiffness of the robot for achieving the accuracy of the surgical instrument to perform bone removal processes. The accuracy necessary for instance for a difficult bone removal process like a cochlear implant is 0.25 mm. Computer Tomography (CT) scan data that indicate where to remove bone have an accuracy of 0.2 mm. That means that a bone removal robot typically must have an accuracy better than 0.05 mm or 50 microns at the active end (tip) of the surgical instrument. The reach of a surgical instrument for bone removal from a skull should be no less than 50×50×50 mm³, preferably larger. To optimize the path of the surgical instrument there should be at least six degrees of freedom and preferably seven. Seven degrees of freedom make it possible for the robot to use its most stiff configuration when choosing the path of the robot during bone removal. To provide seven degrees of freedom the robot according to the invention is equipped with six revolute joints and one prismatic unit. A further advantage can be achieved if the revolute joints near the base, i.e. the first joints are made larger than the others near the last joint. This increases the stiffness of the first joints, which in turn has a disproportionally large effect on the overall stiffness and thus the accuracy of the robot as measured at the tip of the surgical instrument. It is also advantageous to use modular designs for the joints as much as possible, meaning using almost identical joints as much as possible. These requirements result for a robot designed for bone removal of the skull in a typical length between the joints of 75 mm and a diameter of the joints of 71 mm. The first joint is made with a diameter of 120 mm and a length of 125 mm. A robot according to the invention then fits in a box with dimensions of 160×180×200 mm³ and has an ellipsoid reach with radii of 300 and 500 mm, making the robot useful for a number of operations on the skull, like operations on brain tumors, operations on the jaws to remove tumors or to repair (bone around) teeth. Using this inventive setup, the tip of the instrument can be manipulated with an accuracy of better than 50 microns even when bone removal forces are exerted on the tip. The prior art robot reaches an positioning accuracy of 300 microns and shows deflections of 200 microns when a force of 10 N is exerted at the tip of the surgical instrument [B. Bell et al. A self-developed and constructed robot for minimally invasive cochlear implantation. Axta Oto-Laryngologica, 132: 355-360, 2012]. The robot can also be integrated in the operating table. This means that the base of the robot is then part of the operating table.

Preferably the base of the robot comprises a slewing rotational unit, where the slewing unit has its rotation axis perpendicular to the headrest, where the robotic arm is connected to the rotating part of the slewing unit and the headrest is located on top of the stationary part of the slewing unit so that the surgical instrument can rotate around the patient's skull and where the slewing unit has a clamping mechanism to lock the slewing unit with the robotic arm in a desired position. The slewing unit with the headrest is located next to the first joint. This enables the robot to rotate around the patient's skull, i.e. it enables the robot to change the angle of approach or makes it possible to remove the robot out of the way in emergency situations. Preferably a disk brake clamp is used to lock the slewing unit with the robot in a desired position for bone removal.

The slewing unit can also be automated with a motor, encoders and possibly also an automated brake. This would add an extra degree of freedom to the robot.

Preferably the robotic arm is fixated to the base using sliding fitting dowels and releasable fixing means so that the robotic arm can be removed from and reconnected to the headrest and the base in a repeatable way with high accuracy. This makes it possible to remove the robotic arm to enable intra-operative (so during a surgical procedure) CT scans of or manual operations on the skull. After the scan or the manual operation that part of the robot can then be reconnected and the robot can take over the bone removal without recalibrating. In case of a hybrid operation room, i.e. one with the possibility to do inter-operative CT scans, the base of the robot with the headrest and the slewing unit are made from a radiolucent material, so that the base does not interfere with the scans.

The headrest of the robotic instrument comprises fixation components to fix the skull of the patient to the headrest. During bone removal procedures on the skull it is important that the skull is in a fixed position. It is known to fix the skull by pressing it against the headrest manually, i.e. with the hand of a surgeon, by using a skull clamp (Mayfield clamp) or by using pneumatic cushions. However, a skull clamp fixation is relatively invasive for the patient (using three large sharp pins into the front and back of the skull, penetrating the skin and leaving scars (also at the forehead)). A skull clamp also requires relatively much space and reduces the accuracy for image-guided robotic procedures. Pneumatic cushions still allow too much motion of the skull, so that the required accuracy for bone removal with a robot is difficult to obtain.

According to the invention the fixation components comprise a ring upon which the skull of the patient can rest, a preloaded fixation strap that goes around the skull and is connected to the headrest and a fixation plate that fits around part of the skull and is fixated to the skull with at least two bone screws and where the plate can be fixated to the headrest. Here, ring means a rounded shape such as a circular or ellipsoid shape or an open ring like a horseshoe shape. The fixation components fixate the skull of the patient in all six degrees of freedom in a less invasive way, but still with sufficient rigidity to provide safety, accuracy and stability throughout the entire operation. Moreover using these components to fixate the skull, it is possible to have sufficient space for the robot to move around the operating area, while it is also possible to have a sterile layer between robot with head fixation unit and the patient.

If three or more bone screws are used a further advantage of the bone screws is that they can also be used as fiducial markers for registration. This means they provide the reference data for coupling of patient image data, such as CT data, to the patient on the operating table.

Revolute joints of a typical bone removal robot comprise an harmonic drive, where the harmonic drive has an incoming shaft and a flex spline coupled to an outgoing shaft. Harmonic drives are (almost) not backdrivable, i.e. that the input (motor side of a) shaft will not move if a (relatively small) force or torque is applied at the output shaft. Being not or only backdrivable in a limited way can be dangerous if one or multiple humans are close by and/or want to interact with the robot, for safety, manual adjustability and in emergency situations. Sometimes as a solution, an extra device is placed after the harmonic drive gearbox, for example a (friction) clutch, to make safety, manual adjustability and actions in emergency situations possible. However, this makes the robot design larger and heavier, deteriorating the robot's performance. According to the invention an outgoing flange of the flex spline is coupled to a flange of the outgoing shaft via a friction clutch and a decoupling mechanism for the friction clutch, so that the flex spline can be coupled or decoupled from the outgoing shaft. This makes a decoupling of the outgoing shaft and quick movement of the outgoing shaft possible in emergency situations. By using friction force between the outgoing flange surface and the surface of the outgoing shaft to couple and uncouple the outgoing shaft a very compact design is possible, so that the performance of the joint is not affected. This compact design also does not increase the minimal distance between the joints. Thus it does not affect the stiffness of the joint.

Often during multiple tasks of the robot, not all degrees of freedom (moving axes) have to be used, i.e. actively controlled. It is known if less moving axes are required than the robot has available, to hold the axes which are not used, as still as possible using active control. However, due to internal forces/torques, interaction forces/torques and vibrations, these axes cannot be held completely still. The active control gives a relatively low stiffness, therefore resulting in an extra error at the removal tool tip. Moreover, extra heat is generated by the actively controlled axes. Heat generation can be reduced and the accuracy at the tip of the removal tool can be improved if the non-used axes can be locked with a mechanical brake, omitting the need for active control. Moreover, such a mechanical brake improves the stiffness for each joint. In summary mechanical stiffness is larger than servo stiffness. Some mechanical locks already exist, but most of them have unwanted limitations. They are large and/or heavy. They can only lock on discrete positions, while often locking on every position is desired. They are only one-directional (one works one-way), and not bi-directional. They have backlash (play), which is undesirable or they require a high actuation force.

According to the invention a revolute joint has a locking mechanism where an outgoing shaft of the joint is surrounded by a brake ring fixed to a housing of the joint, where the brake ring is surrounded by a actuation ring provided with wedges on its inner diameter and rollers associated with the wedges, where the rollers are located in between the actuation ring and the brake ring, where the actuation ring can be rotated so that the wedges exert forces on their associated rollers whereby the rollers squeeze the brake ring on the outgoing shaft, thus using friction between brake ring and outgoing shaft to lock the outgoing shaft to the housing of the joint. To allow maximum choice of which joints to use during bone removal, preferably all or most of the revolute joints have a locking mechanism.

This locking mechanism according to the invention provides a very compact design that can lock the outgoing shaft to the housing of the harmonic drive and thus to the ingoing axis. The use of the wedging principle with a brake ring provides a very low actuation force and a large holding force. Moreover the minimal distance between the joints is not affected by the lock, thus not decreasing the stiffness of the joint.

It is known to guide the surgical instrument in bone removal using imaging scan data taken previous to the bone removal process. The robot is then guided along a path determined with the scan data. According to the invention the prismatic unit comprises encoder modules to measure the displacement of the surgical instrument and each revolute joint comprises encoders to measure the rotation of the revolute joint. The robot has two encoders per joint for safety and redundancy. The prismatic unit is able to make a limited linear movement, depending on the prismatic unit used, whereas the revolute joints have unlimited range. Using the encoders the guidance system of the robot can accurately and safely guide the surgical instrument along the path determined with the scan data. A force sensor can be used between the prismatic unit and the last revolute joint. The force sensor is an extra safety feature, for instance a change in force during bone removal can indicate a softer or harder bone or tissue, so that the robot can be shut off or guided in a different direction.

The invention also deals with use of the robot according to the invention where the following steps are used to remove bone from a skull:

1. rigidly fixate at least 3 fiducial markers in the vicinity of the intended operating area of the bone of the skull,

2. perform a computed tomography (CT) scan, in which both the operating area and the fiducial markers are visible,

3. import the CT scan data into computer software, from which through image processing desired structures are segmented. The desired structures being at least the fiducial markers, but potentially also other structures such as hard tissue (bone) and soft tissue structures (nerves or blood vessels),

4. make a surgical planning using software to determine the bone volume which has to be removed,

5. perform a path planning using software to calculate the trajectory or trajectories which should be followed by the surgical tool to remove the volume as defined by step 4.

6. transfer the calculated path/trajectory towards individual joint motions of the robot, using an inverse kinematic algorithm of the robot,

7. prepare the operating area for bone removal,

8. clamp the bone of the skull so that it is rigidly attached to the operating area in six degrees of freedom to the base of the robot, or to an intermediate object, which is then again attached to the base of the robot,

9. use the robot's internal encoders and/or an extra apparatus with sensors that is attached to the base of the robot, to determine the locations of all fiducial markers from step 1 to perform a registration, i.e. coupling of CT data from step 2 onto the physical bone from step 8,

10. perform the bone removal task with the robot using the encoders of the joints, at least one for every moving axis, using feedback from the encoders and possibly feedback from a force sensor placed between the last revolute joint and the prismatic unit to determine the location of the tip of the surgical instrument with respect to the patient's data obtained in steps 2 and 3 and check this location with respect to the planned trajectory and adjust the trajectory if needed.

Optionally simulations of the use of the robot can be performed in between steps, for instance in step 6 after the transfer of the path/trajectory to the robot a simulation in software can be performed in which the bone volume (from step 4) removal is simulated using a model obtained from CT scan data in steps 2 and 3. Potentially, also the movement of the robot is simulated, acquired from steps 5 and 6.

After registration in step 9, it might be required to update/recalculate (part of) the path planning and inverse kinematics (steps 5 and 6).

In step 10 using the robot for bone removal the real-time motion of the robot can also be simulated and visualized using the robot's internal encoder data and/or as extra possibility an extra apparatus with sensors that is attached to the base of the robot in combination with the CT data and model from steps 2 and 3.

In step 10 the use of the robot may be interrupted and an extra check performed on the location of the fiducial markers or an extra CT scan may be performed to monitor progress of the bone removal process and to check whether the accuracy and safety can still be guaranteed.

Note that it is also possible to first perform steps 7 and 8 before any other steps are performed.

Also note that step 5 (path planning) might also be imported or adjusted using an external (haptic) device manually.

### DESCRIPTION FIGURES

The invention is further explained with the help of the following drawings.

FIG. 1 gives an overview of the robot for bone removal from the skull of a patient

FIG. 2 shows the headrest and slewing unit in a direction parallel to a patient

FIG. 3 shows the headrest and slewing unit in a direction perpendicular to a patient

FIG. 4 shows two revolute joints connected together

FIG. 5 gives an exploded view of a revolute joint

FIG. 6 shows a friction coupling 41 in the revolute joint

FIG. 7A shows a lock 42 for the revolute joint and 7B a detail of the lock

FIG. 8 gives an overview of the prismatic unit 11 with the surgical instrument 12

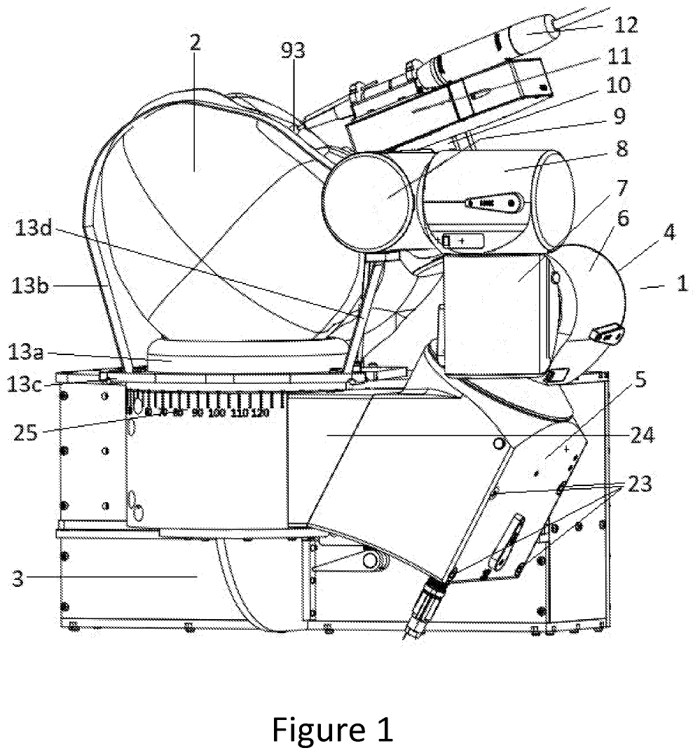

FIG. 1 shows a robot 1 for bone removal from the skull 2 of a patient which robot comprises a base 3 connected to a robotic arm 4 comprising a series of joints (5-11), where the first joint 5 of the series is connected to the base 3 and the last joint 11 of the series is connected to a surgical instrument 12, so that the series of joints 5 to 11 provide degrees of freedom on different axes to the surgical instrument 12, which robot 1 is provided with a headrest 13 (13a,b,c,d,e) for the skull 2. According to the invention the headrest 13 is directly fixated to or is integrated in the base 3 of the robot 1, next to the first joint 5 of the series.

Preferably the series of joints 5-11 comprise revolute joints that are conceptually orthogonal with respect to each-other, where revolute joints have a distance between the joints that is slightly larger than a maximum diameter of the joints and where the last joint 11 is a prismatic unit connected to the surgical instrument 12. Conceptually orthogonal means that the angle between the joints can vary from 80 to 100 degrees, with a preference for 90 degrees. The joints 5 to 10 have a conceptually orthogonal setup. This orthogonal setup for the joints 5 to 10 means that forces from the surgical instrument 12 to the base 3 can be transferred in a path as linear as possible. The revolute joints 5 to 10 according to the invention have a minimal distance between the joints and a maximal diameter of the joints for increased bending stiffness and reduced inertia. In practice this means that the distance between the joints 5 to 10 is slightly larger than a maximal diameter of the joint. Slightly larger means that the diameter (dimension transverse to the distance between the joints) is between 3 to 8% smaller than the distance. In this practical example of the robot the distance is 75 mm and the diameter 71 mm, i.e. around 5% less than the distance between the joints. The distance and the diameter of the joints are determined by the space the robot is allowed to occupy, the reach required for the surgical instrument and the required stiffness of the robot 1 for achieving the accuracy of the surgical instrument 12 to perform bone removal processes. The accuracy necessary for instance for a difficult bone removal process like a cochlear implant is 0.25 mm. Computer Tomography (CT) scan data that indicate where to remove bone have an accuracy of 0.2 mm. That means that a bone removal robot typically must have an accuracy better than 0.05 mm or 50 microns at the active end (tip) of the surgical instrument. The last joint 11 is a prismatic unit, i.e. a linear moving unit with a stroke of 50 mm, connected to the instrument 12. Of course prismatic units with other strokes: longer or shorter can also be used. The reach of a surgical instrument 12 for bone removal from a skull 2 should be no less than 50×50×50 mm³, preferably larger. To optimize the path of the surgical instrument 12 there should be at least six degrees of freedom and preferably seven. Seven degrees of freedom make it possible for the robot 1 to use its most stiff configuration when choosing the path of the instrument 12 during bone removal. A stiffer configuration means more accuracy at the tip of the surgical instrument 12. To provide seven degrees of freedom the robot according to the invention is equipped with six revolute joints 5 to 10 and one prismatic unit 11. A further advantage can be achieved if the revolute joints near the base, i.e. the first joints 5 and 6 are made larger than the others near the last joint 10. This increases the stiffness of the first joints 5 and 6, which in turn has a disproportionally large effect on the overall stiffness and thus the accuracy of the robot 1 as measured at the tip of the surgical instrument 12. In the embodiment the first joint 5 is made with a larger diameter than the rest of the joints. It is also advantageous to use a modular design for the joints as much as possible, meaning using almost identical joints as much as possible. These requirements result for a robot 1 designed for bone removal of the skull 2 in a typical length between the joints of 75 mm and a diameter of the joints of 71 mm. The first joint 5 is made with a diameter of 120 mm and a length of 125 mm. A robot according to the invention then fits in a space of 160×180×200 mm³ and has an ellipsoid reach with radii of 300 and 500 mm. The depth reached depends on the stroke of the prismatic unit 11, in this case 50 mm. These dimensions make the robot useful for a number of operations on the bone of the skull 2, like operations on brain tumors, operations on the jaws to remove tumors or to repair (bone around) teeth. Using this inventive setup the tip of the instrument 12 can be manipulated with an accuracy of better than 50 microns even when bone removal forces are exerted on the tip.

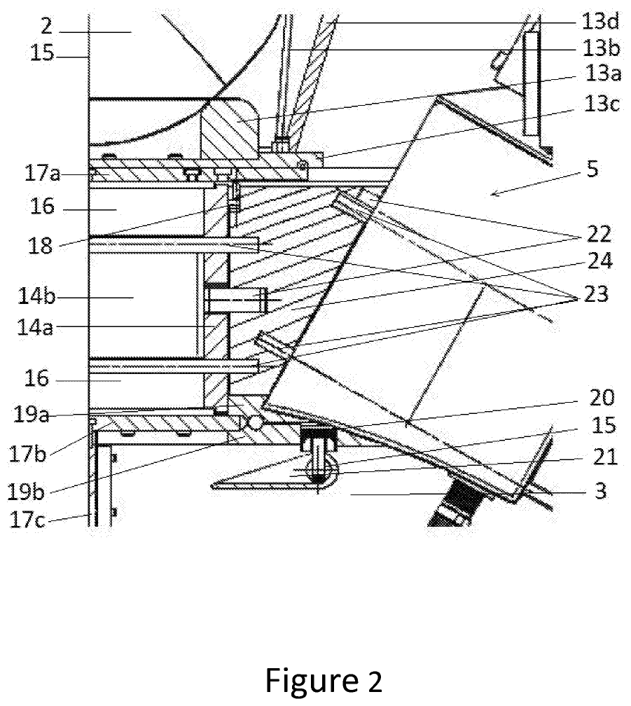

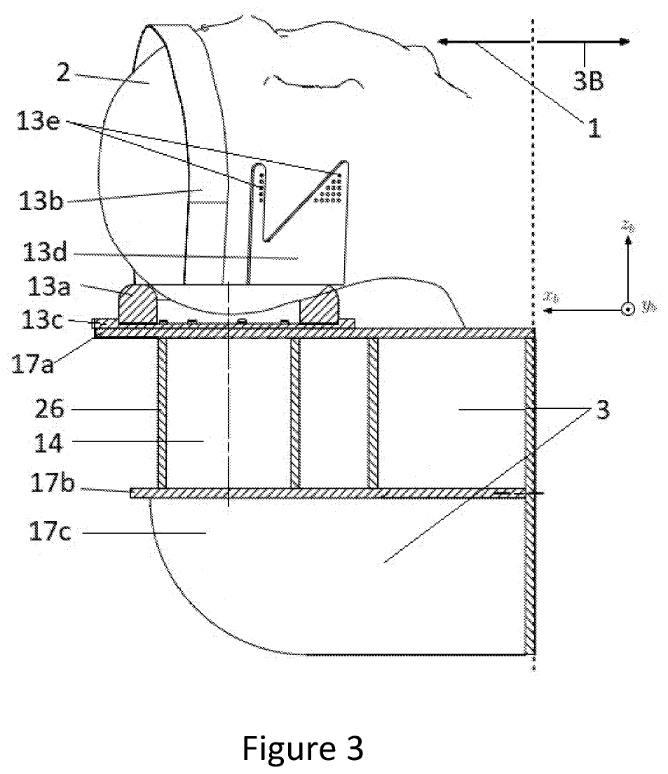

FIGS. 2 and 3 show the base 3 of the robot 1 with the fixation of the robotic arm 4 (see FIG. 1), a slewing unit 14 to enable rotation of the robotic arm 4 around the skull 2 and details of the headrest 13.

In this embodiment the base 3 of the robot 1 comprises a slewing rotational unit 14, where the slewing unit 14 (14a,b) has its rotation axis 15 perpendicular to the headrest 13. The center of the headrest 13 is preferably on the rotational axis 15 of the slewing unit 14. The robotic arm 4 is connected to the rotating part 14a of the slewing unit 14 and the headrest 13 is located on top of the stationary part 14b of the slewing unit 14 so that the surgical instrument 12 can rotate around the patient's skull 2. The slewing unit 14 has a clamping mechanism 15 to lock the slewing unit 14 with the robotic arm 4 in a desired position. A disk brake clamp 15 is used to lock the slewing unit 14 with the robotic arm in a desired position for bone removal. FIG. 2 shows the design of this slewing unit 14 and the disk brake 15. The slewing unit 14 and the disk brake 15 enable a surgeon to convert to conventional manual surgery or change the approach angle in seconds by loosening a disk-brake clamp 15 and rotate the robotic arm 4 manually up to 180° out of the way of the operating area.

Since the headrest 13 is next to the first joint 5, the robot 1 is compact in size and rotation of the robotic arm 4 around the skull 2 does not cause any dangerous situations for the surgeon or other persons near the operation table.

The slewing unit 14 has two plain bearings 16 with axial flanges that guide the rotating part 14a (slewing ring 14a) connected to the robotic arm 4, with respect to stationary part 14b (slewing cylinder 14b) fixed to the headrest 13. The ø90 mm cylinder 14b with 5 mm wall thickness is chosen for relative high stiffness and low mass. In the base 3 there are three top, bottom and vertical plates resp. 17a,b,c with thickness of 6 mm that connect to the cylinder 14b using multiple M4 (polymer) screws. The top plate 17a is connected to the headrest 13. The result is a slewing ring 14a which is guided in three dimensions and can rotate continuously over 180°. A disk-brake mechanism 15, can fixate the slewing unit on every position. Markings 25 in steps of 5° enable detailed positioning (see FIG. 1). Furthermore, a spring enforced, pin-guided clicker system 18 enables repositioning in discrete steps of 10° within ±140 μrad accuracy. Assuming the tool tip of the surgical instrument 12 (see FIG. 1) positioned at a nominal position at radius r=40 mm from the rotation axis 15, this would result in a tool tip repeatability of ±6 μm after a slewing motion. Re-calibration after a temporary slewing motion is therefore not required in most cases. The slewing ring 14a, slewing cylinder 14b and headrest plates 13 are made from 30% Carbon Fiber Reinforced PEEK (CA30), since a radiolucent material is required to enable CT scans with the skull 2 in position and the robotic arm 4 out of the way. PEEK CA30 is chosen over pure carbon fiber solutions due to the increased design freedom to cope with different operating tables, improved manufacturing for bearing surfaces and ability to use screw fixations without metal inserts. Ceramics are not used for their brittleness; being less robust against impacts. PEEK CA30 is chosen over other engineering polymers, due to the relative superior mechanical properties, e.g. a Young's Modulus of 9 GPa, FDA approval and resistance to a wide range of chemicals.

A disk-brake mechanism 15 as shown in FIG. 2 is used, in which the disc is the 6 mm thick plate 17b of the base 3. The disk brake 15 further comprises brake pads 19a,b made from a monolithic elastic material. The brake pads 19a,b are normally clamped against the disc 17b by a disc spring stack 20 and can be opened manually using an eccentric lever 21.

FIG. 2 shows how the robotic arm 4 is fixated to the base 3 using sliding fitting dowels 22 and releasable fixing means 23 so that the robotic arm 4 can be removed from and reconnected to the headrest 13 and the base 3 in a repeatable way with high accuracy. This makes it possible to remove the robotic arm 4 to enable intra-operative (so during a surgical procedure) CT scans of or manual operations on the skull 2. After the scan or the manual operation the robotic arm 4 can be reconnected and the robot 1 can take over the bone removal without recalibrating. As explained before when intra-operative CT scans have to be done the base 3 of the robot 1 with the headrest 13 and the slewing unit 14 are made from a radiolucent material, so that the base 3 does not interfere with the scans.

FIG. 2 also shows how the robotic arm 4, connects to the slewing ring 14a via an intermediate body 24 under an angle of 30° with respect to the axis 15 of the slewing unit 14. The use of an intermediate body 24 offers the advantage that it eases changes of both the nominal position and working area of the robot by replacement of the intermediate body 24 with one with an angle different from 30°. This might be required after feedback from surgeons and for other surgical procedures at the head. The robotic arm 4 connects to the intermediate body 24 via a semi-kinematic coupling using two ø10 mm stainless steel dowel pins 22 which fit into a hole with sliding fit and a slotted hole with sliding fit. Four M6×120 mm screws 23 form the releasable fixing means. The screws 23 are preloaded to reduce hysteresis. A second semi-kinematic coupling enables the connection of the intermediate body 24 with the slewing ring 14a, using three ø10 mm stainless steel dowel pins 22 in the intermediate body and three slotted holes in the slewing ring 14a. Note that three dowel pins 22 with three slotted holes are used instead of two dowel pins with one hole and one slotted hole, therefore posing less demands on proper alignment of the robot during assembly, since this connection will be re-established every time there is an Image Guided Robotic Surgery (IGRBS) procedure using a CT scanner inside a hybrid OR, i.e. one where CT scans are possible. Using H6/h6 tolerances for the dowels and the holes the tip of the surgical instrument 12 can be replaced within ±9 μm during every reconnection. The angle of the tip is prescribed within ±70 μrad accuracy using the H6/h6 tolerances. The headrest 13 is located next to the first joint 5. Due to tilt of the first joint 5 caused by the intermediate plate 24 the distance between the slewing unit 14 at the lower end of the first joint 5 is about one centimeter and the distance at the top of the slewing unit around five centimeters. This means the first joint 5 is next to and as close as the design permits to the headrest 13.

The headrest 13 of the robot 1 comprises fixation components to fix the skull 2 of the patient to the headrest 13. During bone removal procedures on the skull 2 it is important that the skull 2 is in a fixed position. It is known to fix the skull 2 by pressing it against a headrest 13 manually, i.e. with the hand of a surgeon, by using a skull clamp (Mayfield clamp) or by using pneumatic cushions. However, a skull clamp fixation is relatively invasive for the patient (using three large sharp pins into the front and back of the skull, penetrating the skin and leaving scars (also at the forehead)). A skull clamp also requires relatively much space and reduces the accuracy for image-guided robotic procedures. Pneumatic cushions still allow too much motion of the skull, so that the required accuracy for bone removal with a robot is difficult to obtain.

FIGS. 1, 2 and 3 show details of the headrest 13. The fixation components comprise a ring 13a upon which the skull 2 of the patient can rest, a preloaded fixation strap 13b that goes around the skull 2 and is connected to a headrest base plate 13c and a fixation plate 13d that fits around part of the skull 2 and is fixated to the skull 2 with at least two bone screws, where the plate 13d can be fixated to the base plate 13c. The two bone screws are screwed in two of the screw holes 13e in the fixation plate 13d. The headrest base plate 13c is connected to the top plate 17a using screws to keep the structural loop short. Here, the ring shape for headrest ring 13a means a rounded shape such as a circular or ellipsoid shape. The ring 13a can be closed or open as in a horseshoe shape.

Using this headrest 13 for fixating the skull 2 is assumed to be the best compromise between rigidity, stability and invasiveness due to placement of bone screws. This concept assumes a skull 2 to represent an (irregular) sphere. Therefore a ring 13a can be used to constrain the skull 2 in three directions with high stiffness due to a line contact. Preloading is provided using the strap 13b. A skull-fixation plate 13d, which is fixed to the skull 2 using at least two bone screws, is used to constrain the skull 2 in rotational directions. The fixation components of headrest 13 thus fixate the skull 2 of the patient in all six degrees of freedom in a less invasive way, but still with sufficient rigidity to provide safety, accuracy and stability throughout an entire bone removal operation. Moreover using these components to fixate the skull 2, it is possible to have sufficient space for the robotic arm 4 to move around the operating area, while it is also possible to have a sterile layer between robotic arm 4, the headrest 13 and the patient. The skull stiffness in combination with the aforementioned proposed skull fixation method is at least 2·10⁶ N/m, in all directions. Therefore, it can likely be assumed relatively stiff in comparison with the stiffness of tip of the surgical tool 12. If three or more bone screws are used a further advantage of the bone screws is that they can also be used as fiducial markers for registration. This means they provide the reference data for coupling of patient image data, such as CT data to the patient on the operating table.

The robot 1 can be integrated in an operating table. This means that the base 3 of the robot is then part of the operating table. FIG. 3 shows an embodiment where the base 3 of the robot can be fixed to an operating table 3B. The slewing cylinder 14 is fixed in six degrees of freedom to a custom radiolucent headrest 13. The fixation is a combination of a three plates: a T-profile at the bottom made from two plates 17b and 17c and one top horizontal plate 17a. The T-profile fixates with high stiffness due the use of two on-edge plates, but is torsionally compliant. To fix this there is a top horizontal plate 17a, at a distance of 100 mm from the bottom plate 17b. Note, using this second horizontal plate 17a results in a three-times overdetermined design. This, however, increases in-plane stiffness and results in more symmetry with respect to the center of the slewing unit 14. Furthermore, no internal compliance is created to have a closed structure for patient safety and sterility. In this case where the headrest 13 is not integrated in an operation room table, a torsion tube 26 is suggested for increased stiffness before a connection with an operating table 3B is made.

All materials used for the construction in FIG. 3 should be made radiolucent in case CT scans will be made in situ, i.e. with the skull 2 of the patient on the headrest 13.

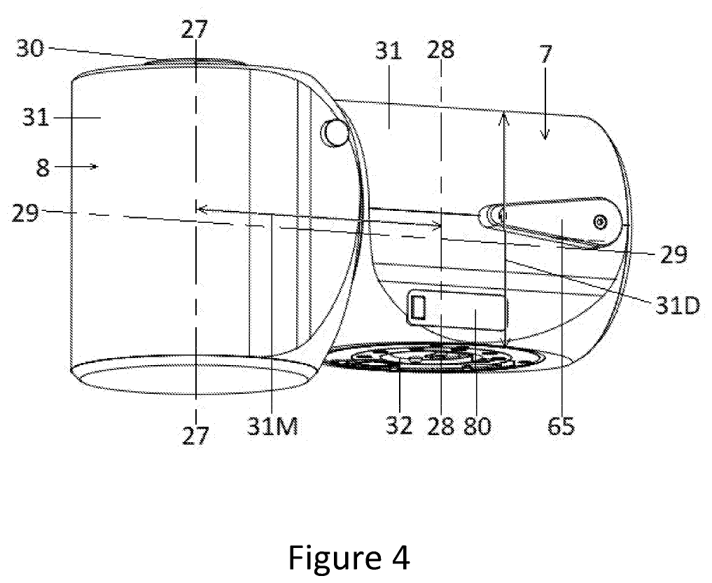

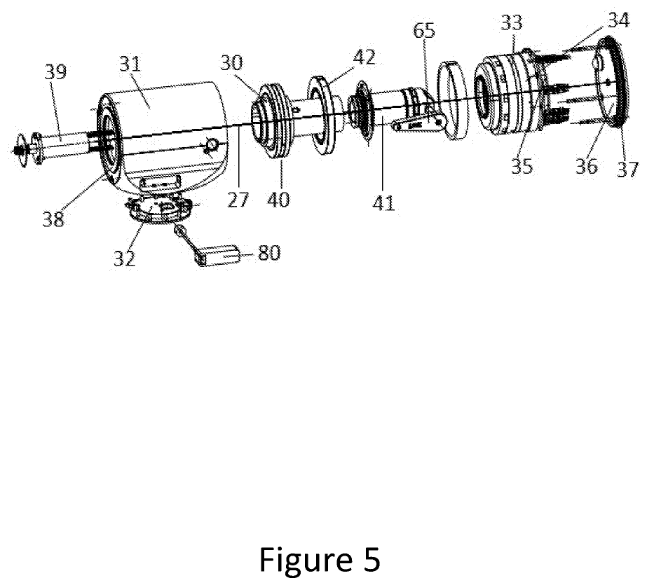

FIG. 4 shows two of the series of orthogonal revolute joints 5 to 10 (see FIG. 1). The design of the joints 6 to 10 is modular (stand-alone) so the joints in FIG. 4 are similar to other joints. For the example of FIG. 4 joints 7 and 8 have been taken. These joints are situated between joints 6 and 9 (not shown). A modular revolute joint according to the invention has an output axis and an input axis placed orthogonal with respect to each other (see also FIG. 5). Joint 7 has an input axis 28 and an output axis 29. Joint 8 has an input axis 29 and an output axis 27. Thus the axis 29 in FIG. 4 is the output axis of joint 7 and at the same time the input axis of joint 8. On the output axis 27 there is an outgoing shaft 30 that can rotate with respect to the housing 31 of the joint. The outgoing shaft 30 is coaxial with axis 27. On the input axis 28 there is a connector 32 that is fixated to the housing 31. The connector 32 is coaxial with axis 28. The outgoing shaft 30 of a joint connects to the connector 32 of a following joint. The connector 32 of a joint connects with the outgoing shaft 30 of a previous joint. In FIG. 4 joint 7 has an input connector 32 that is connected to the output shaft 30 of the previous joint 6 (not shown). The output shaft 30 of joint 7 is connected to connector 32 of joint 8 (not shown in FIG. 4 since hidden behind housing). The output shaft 30 of joint 8 is connected to connector 32 of the following joint 9 (also not shown). The output shaft 30 is provided with a thread M30×1 mm. The connector 32 uses multiple bolts to screw it in the housing 31. The connector 32 has an internal thread M30×1 mm. When connected the M30×1 mm threads on shaft 30 and connector 32 provide a loosening force of 8 kN. Build in the connector there is an extra clamp for fixating the shaft 30 for extra safety. This way multiple revolute joints can be combined to build a serial robotic arm 4 with a desired number of degrees of freedom. FIG. 4 also shows the minimal distance 31M between the joints 7 and 8. This is a distance on axis 29 limited by the axes 27 and 28, i.e. link length between the joints. The diameter 31D of the joints is slightly smaller than this minimal distance 31M so as not get any interference of the joints with each-other when moving the robotic arm 4.

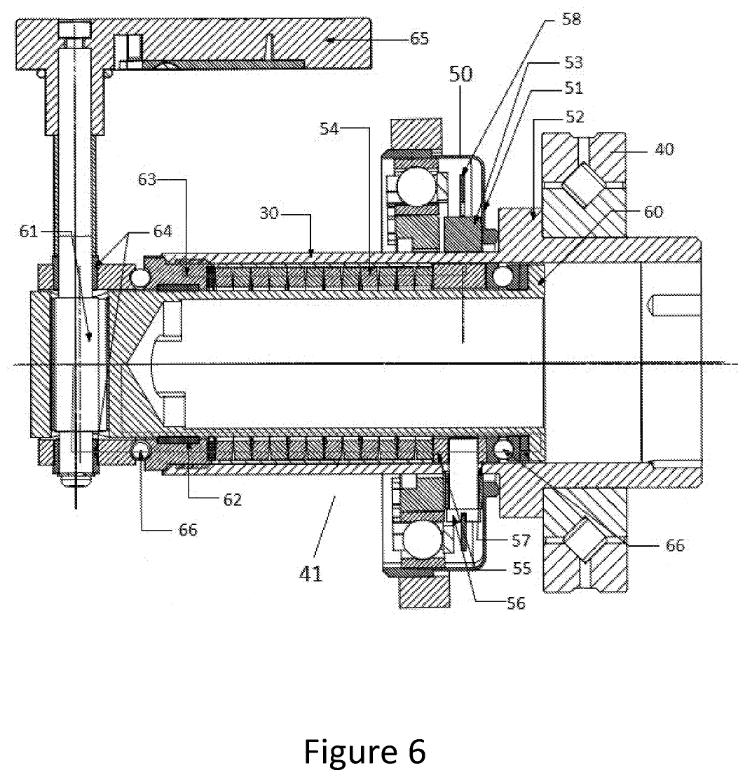

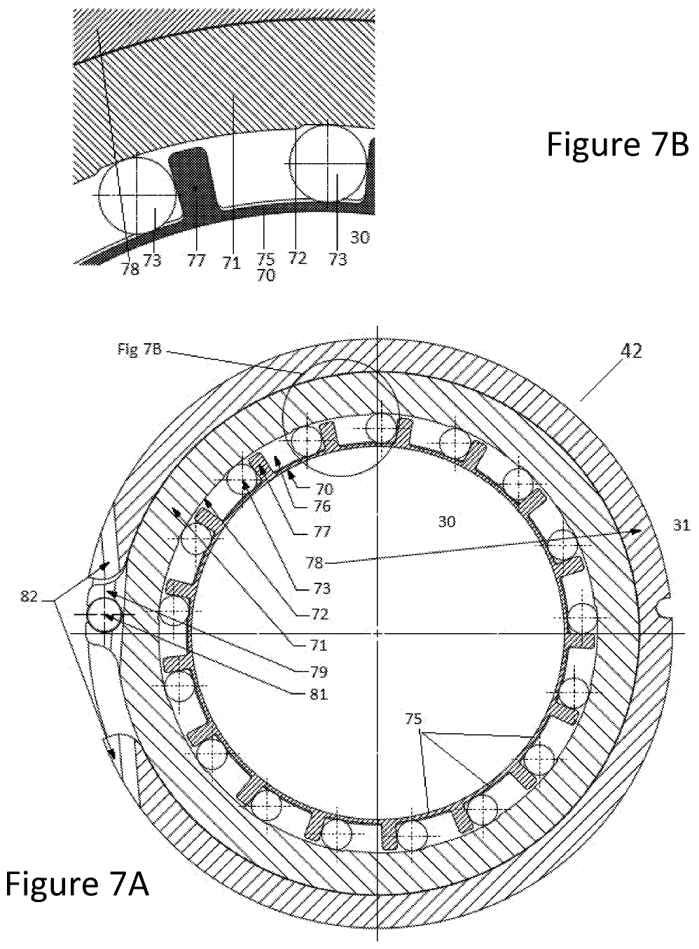

FIG. 5 provides an exploded view of one of the orthogonal revolute joints 5 to 10. According to the invention these joints have a possibility to couple and decouple in and outgoing shafts 30 shown in detail in FIG. 6 and they have a lock on the outgoing shaft 30 shown in detail in FIG. 7.

FIG. 5 shows an exploded view of all subsystems of the joints 5 to 10. Each subsystem is placed concentrically with the output axis 27 and can be assembled from the back. The revolute joint comprises a gearbox 33, a motor 34 that drives an input shaft of the gearbox 33 and an output shaft 30. As gearbox 33 an harmonic drive is used. Harmonic drives are gearboxes with large reductions typical 50 to 100, commercially available from the firm Harmonic Drive. Harmonic drives comprise an input shaft connected to a wave generator that acts upon a flex spline connected to an output shaft. In the revolute joints an harmonic drive 33 with a reduction factor of 100 is used, with the input shaft of the harmonic drive connected to a shaft of the motor 34. The motor shaft is equipped with a 16 bit optical encoder 35. The motor 34 is a brushless direct current (BLDC) motor electrically connected to a printed circuit board 36 mounted on a cover 37. The housing of the motor 34 is fixated to the housing 31 of the joint. The output side of the harmonic drive is connected to output shaft 30 that is equipped with a second magnetic encoder 38 for increased accuracy and safety. The joint is further provided with a slip ring 39 for electrical connections and a cross-roller bearing 40 for the output shaft 30. The cross-roller bearing 40 fixates the outgoing shaft 30 in five degrees of freedom. Cross-roller bearings provide reduced axial length, less weight and inertia as compared to two angular contact roller bearings. They still provide sufficient radial, axial and tilting stiffness to be non-limiting in the structural loop.

The joint according to the invention further comprises a friction clutch 41 with a lever 65 so as to be able to couple and uncouple the output shaft 30 to the outgoing side of the harmonic drive. The joint also comprises a lock 42 with a lever 80 to lock the outgoing shaft 30 to the housing 31.

Due to their large reduction (factor 50 to 100) harmonic drives are almost not back-drivable. Being not back-drivable or only in a limited way backdrivable can be dangerous if one or multiple humans are close by and/or want to interact with the robot, for safety, manual adjustability and in emergency situations. Thus back-drivability of the drivetrain is desired for safety and human-robot interaction.

As a solution to the non-backdrivability, a friction clutch 41 with a manual decoupling mechanism is designed in between the harmonic drive 33 and output axis 30. This clutch 41 enables a torque limiting functionality for safety, the ability for low-force manual take-over, and the prevention of damage to the drivetrain by overload.

The mechanical design of the friction clutch 41 will be described in more detail using FIG. 6. An outgoing flange 51 of the flex spline 50 is coupled to a flange 52 of the outgoing shaft 30 via a friction clutch 41 and a decoupling mechanism for the friction clutch, so that the flex spline 50 can be coupled or decoupled from the outgoing shaft 30. The friction clutch 41 uses two surfaces. One surface is a surface of the flange 52 of the output shaft 30. The second surface is a surface of flange 51, i.e. the endplate of the flex spline 50, which is an axially compliant part and normally used to connect to an output axis of the flex spline 50 of the harmonic drive. These two surfaces can be pressed together under a preload force that can be exerted and removed. The interface between the flanges 51 and 52 thus provides a friction force that holds the output shaft 30 fixed to the flex spline 50. The structural force loop of the clutch mechanism is designed to be short in both engaged (top of FIG. 6) and disengaged (bottom of FIG. 6) positions. One side of the flex spline flange 51 is pressed against the output axis flange 52 using a push ring 53. The push ring 53 is axially preloaded using a spring 54 which provides the normal force. However, there is no volume for the spring available behind the push ring 53, since the components of the harmonic drive and the motor (both not shown) are located there. Therefore the spring 54 is placed inside the output shaft 30. The push ring 53 is connected with an inner push ring 55, placed inside the output shaft 30, using three ø3.5 mm hardened pins 56 which protrude through three axial slotted holes 57 in the output axis. A retaining ring 58 prevents the three pins 56 from falling out. The inner push ring 55 is axially guided inside the output axis using three contact surfaces from PTFE tape for reduced friction. As a result, two sets of friction interfaces are available for the friction clutch 41: One interface formed by surfaces of flanges 51 and 52, the other by a the opposite surface of flange 51 and a surface on the push ring 53. This lowers the required normal force, and therefore lowers the volume of the spring 54 with a factor two. The contact surfaces of both the flex spline flange 51 and push ring 53 are reduced to increase surface pressure and therefore reduce hysteresis.

To decouple the friction clutch 41, a pull tube 60 is used to remove the normal force from the flex spline 50 by axially compressing the spring 54. This axial motion is provided by rotation of an eccentric axis 61 which houses inside the back of the pull tube 60. A modified plain bearing 62, is placed inside a groove in a preload screw 63 for the spring 54. This bearing 62 acts as linear guidance for the pull tube 60. The eccentric shaft 61 is used to move the pull tube 60. An eccentricity of 0.5 mm provides a ratio to reduce the force required to decouple the friction clutch 41. The eccentric axis 61 is guided using two plain bearing bushes 64 from hardened steel in a hardened circular housing, which surrounds the pull tube 60 to keep the force path short. Hysteresis between eccentric shaft 61 and pull tube 60 is reduced by enclosing the eccentric shaft 61 with the pull tube 60, since the pull tube does not rotate with respect to the force vector, while the eccentric shaft 61 does. The eccentric shaft 61 can be operated manually by flipping a lever 65. The eccentric mechanism 61 is designed to be at the top dead center at decoupled position, keeping the lever 65 and eccentric 61 in position by friction. The lever has a length of 35 mm, resulting in a maximum actuation force of 45 N when a friction coefficient μ=0.2 is assumed. Two full-complement thrust bearings 66 are placed in between the preload spring 54, the pull tube 60 and the circular housing, since the preload spring and output axis rotate with respect to the housing whilst the eccentric axis does not.

The friction clutch 41 makes a decoupling of the outgoing shaft 30 and quick movement of the outgoing shaft 30 possible in emergency situations by rotating lever 65. By using friction force between the outgoing flange surface 51 of the flex spline 50 and the surface 52 of the outgoing shaft 30 to couple and uncouple the outgoing shaft 30 a very compact design is possible, so that the performance of the joint is not affected by the friction clutch 41. This compact design also does not increase the minimal distance 31M between the joints. Thus it does not affect the stiffness of the joint.

FIG. 7 shows the design of a locking mechanism 42 on the outgoing shaft 30 of a joint. Here FIG. 7B shows a detail and FIG. 7A the overall design.

The revolute joints 5 to 10 have a locking mechanism 42 where an outgoing shaft 30 of the joint is surrounded by a brake ring 70 fixed to a housing 31 of the joint, where the brake ring 70 is surrounded by a actuation ring 71 provided with wedges 72 on its inner diameter and rollers 73 associated with the wedges 72, where the rollers 73 are located in between the actuation ring 71 and the brake ring 70, where the actuation ring 71 can be rotated so that the wedges 72 exert forces on their associated rollers 73 whereby the rollers 73 squeeze the brake ring 70 on the outgoing shaft 30, thus using friction between brake ring 70 and outgoing shaft 30 to lock the outgoing shaft 30 to the housing 31 of the joint. To allow maximum choice of which joints to use during bone removal, preferably all or most of the revolute joints 5 to 10 have a locking mechanism 42. The lock 42 fixates the moving output shaft 30 with respect to the housing 31 of the joint. To achieve relative fixation, multiple friction surfaces 75, called patches, are used on the brake ring 70. Each friction patch 75 is thin, enabling elastic deformation in radial direction. Radial deformation is provided by rollers 73 and an actuation ring 71 with wedges 72. The rollers 73 also act as guidance for the actuation ring 71, eliminating the need of an extra bearing. The brake ring 70 is fixated to the housing 31 of the joint with two plates 76 using battlements 77 on the brake ring 70. These plates 76 are placed in a sandwich setup for symmetry and increased stiffness. Moreover, the plates 76 enclose the rollers 73 and protect the raceways from debris from the outside world. An outer ring 78 acts as spacer for the plates 76 and connects to the housing 31. Both plates 76 can be fixated to the outer ring 78 using friction, adhesive bonding or spot welding (with minimum deformation due to heat gradients). Of those, friction, which was observed to suffice during tests, was chosen since this simplifies assembly and disassembly. A shoulder 79 is placed at the outside of the actuation ring 71 to create space for an actuation rod 80 (shown in FIG. 5) which can make a tangential movement. A roller with internal thread 81 fits inside the shoulder 79 and is used to grasp a threaded rod at a fixed radius. The outer ring 78 is opened to accommodate the shoulder. Moreover, slots 82 are created in the outer ring 78 to make clearance for the actuation rod 80. Multiple rollers 73 and friction patches 75 are used to increase the locking torque. The number of friction patches 75 on the brake ring 70 is limited by roller 73 size and travel; the latter being half of the stroke of the actuation ring 71. When the lock 42 is not engaged, there is a constant gap of approximately 10 to 15 μm between the brake ring 70 and output shaft 30. As a result, there is no friction and therefore no virtual backlash on the output motion. A tangential movement of, and force on the actuation ring 71 will engage the lock 42. First, the tangential movement results in combined tangential rolling and radial movement of the rollers 73, resulting in elastic deformation of the friction patches 75 until the output shaft 30 is hit. Note the same radial force is exerted on the actuation ring 71, but the stiffness of the actuation ring 71 is a few orders higher, resulting in a negligible deformation compared to the deformation of the friction patches 75. When the tangential force is increased after hitting the output shaft 30, each roller 73 exerts a normal force in radial direction on both the brake ring 70 and actuation ring 71. The radial forces between the output shaft 30 and brake ring 70 (friction patches 75) will generate friction and therefore lock the shaft 30. If the tangential actuation force is removed again, the friction patches 75 will exert a negative radial force on the rollers 73. As a result, the friction patches 75 are designed to elastically return to their initial state, whilst pushing the rollers 73 and actuation ring 71 back.

This locking mechanism 42 provides a very compact design that can lock the outgoing shaft 30 to the housing 31 of the joint and thus to the ingoing shaft. The use of the wedging principle 72 with a brake ring 70 provides a very low actuation force and a large holding force. Moreover the minimal distance 31M between the joints is not affected by the lock 42. Thus not decreasing the stiffness of the joint.

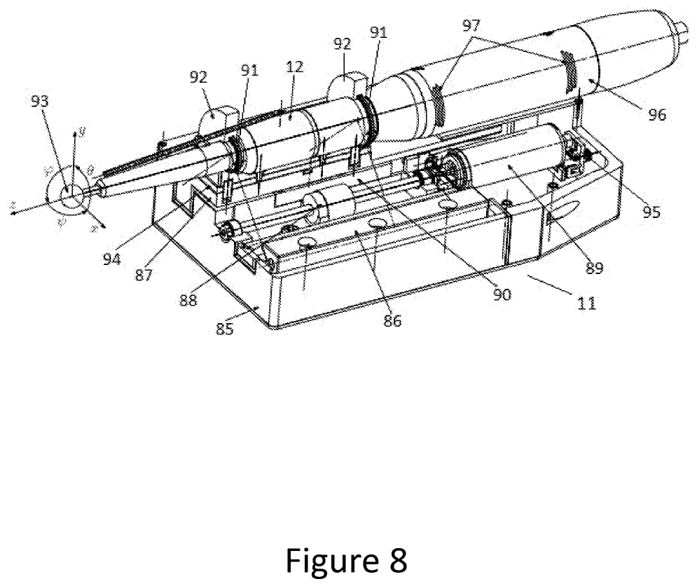

FIG. 8 shows a partially opened up view of the prismatic linear unit 11 and the surgical instrument 12 (see also FIG. 1). The prismatic linear unit 11 has an open box frame 85 with anti-torsion tube, i.e. an extra internal intermediate plate parallel to the bottom plate of the unit. Linear cross-roller bearings 86 guide the surgical instrument 12 on a carriage 87, which is actuated using a leadscrew 88 and a brushless DC motor 89. Absolute position is measured both at the moving carriage 87 and BLDC motor using an absolute encoder system 90.

A standard surgical instrument 12 is clamped at two points by compression of two elastomeric rings 91 in a tool-adapter 92. The surgical instrument carries a bone removal tool 93, such as a cutter or a drill. This tool-adapter 92 can be interchanged to hold other surgical instruments 12 and can be made disposable or sterilizable by an autoclave.

Elastomeric rings 91 are chosen to increase damping at the expense of stiffness close to the surgical instrument 12, to reduce unwanted disturbance forces going into the robotic arm 4 as close as possible to the source. These (high-frequency) disturbances, which are a result of bone removal dynamics such as occur during milling or drilling, are unwanted, since they can excite vibrations in parts of the robotic arm 4. The stiffness of the compressed rings 91 is calculated to be at least a factor higher than the stiffness of the tool 93 and its holder in the surgical instrument 12 such as a combination of collet with a cutter or drill. Therefore elastomeric rings 91 are not assumed to be the limiting factor in the structural loop. Two clamps 92 with grooves are used to obtain radial compression of the rings 91.

The clamps do not impede tool 93 changes, which can be done within a minute. The first fixation point of the tool adapter 92 is chosen as close as possible to the tool 93, but with a distance of 50 mm to have sufficient reach inside a patient. If a depth of 75 mm is required during surgery, the tool 93 should be changed to a tool with a longer shaft. The second fixation point is placed at 45 mm from the first to keep the tool-adapter 92 compact and light.

Moreover, the motor 96 of the surgical instrument 12 has cooling grooves 97 which should not be blocked by a clamp. The surgical instrument 12 can be loaded with high repeatability, since a flange on the adapter 92 acts as axial stop for a flange on the surgical instrument 12. The axial distance of the tool 93 with respect to the tool adapter 92 can be measured with 2 μm accuracy on forehand using a micrometer gauge. The connection between tool-adapter 92 and linear carriage 87 is made using a semi-kinematic connection with sliding fit dowel pins. In between the tool adapter 92 and the carriage 87 a sterile plate 94 is fitted.

The sterile plate 94 is used to enable placement of a sterile draping with a thickness of 50 μm between robotic arm 4 and surgical tool 93, while being able to change surgical tools 93 during surgery without sacrificing sterility. The draping covers the remaining robotic arm 4 and headrest 13, to prevent bidirectional contamination between robot 1 and patient. The 50 μpm thick draping also acts as a thin gasket, since compression between edges on the adapter plate 92 and draping will result in a sealing. The tool-adapter 92 is fixated to the sterile plate using two M2 screws. The sterile plate 94 is fixated to the carriage 87 using four M2.5 screws which are also used for fixation of the linear bearings 86. The carriage 87 is guided over a stroke of 50 mm using two linear bearings 86 with running cross-roller cages. Rollers are used instead of balls, because they can withstand 50 times larger loads and have a factor 10 higher stiffness at comparable size and maximum load. The bearing poles (being intersection ‘points’ of the contact-lines of the rollers) intersect with the tool axis. This results in pure forces and no bending moments on the guidance. One linear bearing 86 acts as main guide rail, while the other is used as secondary guide rail.

In between guide rails, a 03.3 mm lead-screw 88 with 1.22 mm pitch and anti-backlash (AB) nut is used for actuation in axial direction. A leadscrew 88 is chosen for its axial stiffness and its possibility to back-drive in emergency situations. The leadscrew 88 is attached to the linear frame using two preloaded angular ball bearings in O-arrangement. Next to the leadscrew, an encoder system 90 measures the absolute displacement between linear carriage 87 (therefore the surgical tool tip 93) and the linear frame 85 with a resolution of 0.3 μm and accuracy of ±3 μm over 1 m. The encoder ruler is assembled against an x and z edge on the carriage 87, defining all in-plane degrees of freedom while having the possibility to cope with thermal expansion differences between ruler (stainless steel) and carriage 87 (aluminum).

A BLDC motor 89 is used to drive the leadscrew 88 via a direct-drive setup for high dynamic performance without backlash. A 12-bit magnetic absolute encoder 95 is added on the back of the BLDC motor 89 for redundancy (safety) and the ability to control the motor with high efficiency at low speeds. The leadscrew-motor combination 88, 89 is able to deliver a continuous axial force of 15 N, with speed up to 400 mm/s. Axial forces up to 100 N can be provided at lower speeds and in short term operation. The maximum (no-load) speed is 700 mm/s, which enables surgical tool retraction within 0.1 s in case of emergencies. The leadscrew 88 is connected to the motor 89 using an elastic coupling with four tangential struts. Two struts are connected to the motor 89 and two struts are connected to the leadscrew 88. The linear frame is designed to resemble a closed box for a lightweight structure with high rigidity. However, an opening at the top of the frame is required for the 50 mm stroke of the carriage 87 and tool 93, resulting in an open box structure. An anti-torsion tube, i.e. an extra intermediate plate parallel to the bottom, is enclosed to increase torsional stiffness of the open box frame. The bottom plate, i.e. one of the endplates of the tube acts as connection plane/plate to the previous revolute joint 10. On this plate, the guide rails, leadscrew 88 and readheads of the encoder 90 are assembled for in-plane stiffness between components.

The linear frame 85 is connected to a six degrees of freedom force-torque (F/T) sensor, which can measure forces and torques exerted on the surgical instrument 12. The F/T sensor is positioned as close as possible to the surgical instrument 12, can measure in-plane forces up to 80 N with a resolution of 0.03 N and can measure in-plane torques up to 2 Nm with a resolution of 0.5 Nmm. The sensor, made from a titanium alloy, has a mass of 0.033 kg and can be overloaded up to ±1500 N and ±30 Nm, resulting in a robust solution. The force sensor is connected to the output shaft 30 of the previous modular revolute joint 10.

It is known to guide the surgical instrument in bone removal using CT scan data taken previous to the bone removal process. The robot is then guided along a path determined with the scan data.

Reference is made to FIGS. 1, 5 and 8. For steering the robot 1 the path is determined using the encoder modules 90, 95 of the prismatic unit 11 and the encoders 35 and 38 of the revolute joints 5 to 10 of robot 1. The encoder 90 and the encoder 95 at the back of the motor 89 of the prismatic unit 11 give the displacement of the surgical instrument 12. Each revolute joint 5 to 10 comprises encoders 35, 38 that give the rotation of the revolute joints 5 to 10. The robot 1 has two encoders per joint 5 to 11 for safety and redundancy, one an optical encoder and the other a magnetic encoder. The prismatic unit 11 is able to make a limited linear movement, depending on the prismatic unit used, whereas the revolute joints 5 to 10 have infinite range. Using the encoders the guidance system of the robot 1 can accurately and safely guide the surgical instrument 12 along the path determined with the scan data. The force sensor can be used between the prismatic unit 11 and the last revolute joint 10. The force sensor (F/T) is an extra safety feature, for instance a change in force during bone removal can indicate a softer or harder bone or tissue, so that the robot can be shut off or guided in a different direction.

Before a surgical procedure can take place, at least three miniature bone-attached fiducial markers are placed in the patient's skull 2 around the intended surgical work area. Local anesthesia is induced. Next, hi-resolution CT images or images using another scanning technique are made. Semi-automatic image segmentation algorithms are used to find important 3D structures, like bone structures, nerves, blood vessels or organs. Next, the surgeon plans the procedure and determines the to-be-removed bone and structures. A path planning procedure (identical to a trajectory generation) and simulation is performed to estimate automated robotic surgery feasibility and potential risks. Note an approximated position of the robotic arm 4 with respect to the patient's skull 2 at the OR table is used, since the precise location is not yet known.

At the start of the surgical procedure, the skull 2 is fixated with respect to the base 3 of the robot 1 using the headrest 13. A robotic registration is performed, in which the robot 1 acts as a coordinate measurement machine (CMM) to determine the position of the fiducial markers with micrometer accuracy. Now, the path planning can be updated, since the position of the skull 2 of the patient is known with respect to the robotic arm 4. This path planning can be performed offline, since the fixation of the skull 2 with respect to the robotic arm 4 is assumed to be relatively stiff and stable. Joint space control with feed forward is assumed to be sufficient, since the robot 1 can be analyzed as semi-static. Here, the output from the path planning results in the required trajectory of the tip of tool 93. A high level controller converts the motions of the tool tip 93 to seven individual joint reference signals.

Due to a redundant number of degrees of freedom, the reference signals can be optimized for e.g. minimal joint velocities and accelerations, the avoidance of patient- and self-collisions, and for trajectories with maximum stiffness, thus maximum precision. Seven individual single-input single-output (SISO) feedback controllers are used, controlling the position (and speed) of each joint 5 to 11 independently from the others in real time. These are closed in the loop using sequential loop-shaping. Via an EtherCAT bus system, all control signals and measurements are communicated. Each individual joint 5 to 11 (i.e. module) contains local electronics and firmware 36 to perform motor commutation, motor control and input/output (I/O) at approximately 20 kHz. Individual joint measurements, and a tip force measurement, are fed back for low level feedback control and might be used for reference compensation in the high level controller. This high level controller comprises of forward and inverse kinematic and dynamic models, safety checks, and singularity and collision avoidance algorithms.

The inverse dynamics model can be used to determine a feedforward control action to reduce tracking errors. This model should at least contain harmonic drive non-linear stiffness, weight and friction compensation, being the dominant factors. Note the high level controller can be implemented offline in case of Image Guided Robotic Sculpture (IGRBS).

However, if implemented in real-time, joint reference compensation in the high level controller is also possible to reduce the position error. Multi-input multi-output (MIMO) controllers can be an alternative, since they offer inverse dynamics control and robust control. These MIMO controllers should be able to improve performance even more, since all non-linear terms are taken into account and only the point of interest, i.e. the tool tip 93 is controlled. Finally, force control such as compliance control can be implemented using less mature kinematic and dynamic models. In this case the interaction force at the tip 93 is controlled (which can be measured using the six degrees of freedom force/torque sensor).

Although this will result in an increased safety, performance in terms of positional accuracy is assumed to be less than using other control schemes.

Although pre-operational CT data are used to autonomously control the robot 1, supervisory feedback is also provided by vision of the surgeon using existing microscopes, which can be replaced by 3D cameras with augmented reality in the future.

Besides autonomous image guided motion using offline-trajectory calculation, it is possible to let a surgeon steer the robot 1 using a haptic device. In that case, the patient-specific map can still be used for safety, potentially constraining the surgeon's motions close to vital structures. This would however require the high level controller to run in real-time, requiring more computing power and use of efficient algorithms.

The invention also deals with use of the robot according to the invention where the following steps are used to remove bone from a skull:

1. rigidly fixate at least 3 fiducial markers in the vicinity of the intended operating area of the bone of the skull 2,

2. perform a computed tomography (CT) scan, in which both the operating area and the fiducial markers are visible,

3. import the CT scan data into computer software, from which through image processing desired structures are segmented. The desired structures being at least the fiducial markers, but potentially also other structures such as hard tissue (bone) and soft tissue structures (nerves or blood vessels),

4. make a surgical planning using software to determine the bone volume which has to be removed,

5. perform a path planning for the surgical tool 93 of a robot 1 using software to calculate the trajectory or trajectories which should be followed by the surgical tool 93 to remove the volume as defined by step 4,

6. transfer the calculated path/trajectory towards individual joint motions of the robot 1, using an inverse kinematic algorithm of the robot 1,

7. prepare the operating area for bone removal,

8. clamp the bone of the skull 2 which is rigidly attached to the operating area in six degrees of freedom with the use of the headrest 13 to the base of the robot 1,

9. use the robot's internal encoders 35, 38, 90, 95 and/or an extra apparatus with sensors that is attached to the base 3 of the robot 1, to determine the locations of all fiducial markers from step 1 to perform a registration, i.e. coupling of CT data from step 2 onto the physical bone from step 8,

10. perform the bone removal task with the robot 1 using the encoders 35,38, 90, 95 of the joints 5 to 11, at least one for every moving axis, using feedback from the encoders and possibly feedback from a force sensor placed between the last revolute joint and the prismatic unit to determine the location of the tip 93 of the surgical instrument 12 with respect to the patient's data obtained in steps 2 and 3 and check this location with respect to the planned trajectory and adjust the trajectory if needed.

1. A robot for bone removal from the skull of a patient which robot comprises a base connected to a robotic arm comprising a series of joints, where the first joint of the series is connected to the base and the last joint of the series is connected to a surgical instrument, so that the series of joints provide degrees of freedom on different axes to the surgical instrument, which robot is provided with a headrest for the skull, wherein the headrest is directly fixated to or is integrated in the base of the robot, next to the first joint of the series.

2. The robot according to claim 1, wherein the series of joints comprises revolute joints that are conceptually orthogonal with respect to each-other, where revolute joints have a distance between the joints that is slightly larger than a maximum diameter of the joints, where the last joint is a prismatic unit connected to the surgical instrument.

3. The robot according to claim 1, wherein the base of the robot comprises a slewing rotational unit, where the slewing unit has its rotation axis perpendicular to the headrest, where the robotic arm is connected to the rotating part of the slewing unit and the headrest is located on top of the stationary part of the slewing unit so that the surgical instrument can rotate around the patient's skull and where the slewing unit has a clamping mechanism to lock the slewing unit with the robotic arm in a desired position.

4. The robot according to claim 1, wherein the robotic arm is fixated to the base using sliding fitting dowels and releasable fixing means so that the robotic arm can be removed from and reconnected to the headrest and the base in a repeatable way with high accuracy.

5. The robot according to claim 1, wherein the headrest comprises fixation components to fix the skull of the patient to the headrest wherein in that the components comprise a ring upon which the skull of the patient can rest, a preloaded fixation strap that goes around the skull and is connected to the headrest and a fixation plate that fits around part of the skull and is fixated to the skull with at least two bone screws and where the plate can be fixated to the headrest.

6. The robot according to claim 5, wherein at least 3 bone screws are used that can also serve as fiducial markers for imaging scan data.

7. The robot according to claim 2, wherein a revolute joint comprises a harmonic drive, where the harmonic drive has an incoming shaft and a flex spline coupled to an outgoing shaft, wherein an outgoing flange of the flex spline is coupled to a flange of the outgoing shaft via a friction clutch and a decoupling mechanism for the friction clutch, so that the flex spline can be coupled or decoupled from the outgoing shaft.

8. The robot according to claim 2, wherein a revolute joint has a locking mechanism where an outgoing shaft of the joint is surrounded by a brake ring fixed to a housing of the joint, where the brake ring is surrounded by an actuation ring provided with wedges on its inner diameter and rollers associated with the wedges, where the rollers are located in between the actuation ring and the brake ring, where the actuation ring can be rotated so that the wedges exert forces on their associated rollers whereby the rollers squeeze the brake ring on the outgoing shaft, thus using friction between brake ring and outgoing shaft to lock the outgoing shaft to the housing of the joint.

9. The robot according to claim 1, wherein the surgical instrument can be guided using imaging scan data taken previous to the bone removal process, wherein the prismatic unit comprises encoder modules to measure the displacement of the surgical instrument and each revolute joint comprises encoders to measure the rotation of the revolute joint.

10. A method for bone removal from the skull of a patient by a robot that comprises a base connected to a robotic arm comprising a series of joints, wherein the first joint of the series is connected to the base and the last joint of the series is connected to a surgical instrument, so that the series of joints provide degrees of freedom on different axes to the surgical instrument, which robot is provided with a headrest for the skull, wherein the headrest is directly fixated to or is integrated in the base of the robot, next to the first joint of the series, comprising the following steps:
1. rigidly fixate at least 3 fiducial markers in the vicinity of the intended operating area of the bone of the skull,
2. perform a computed tomography (CT) scan, in which both the operating area and the fiducial markers are visible,
3. import the CT scan data into computer software, from which, through image processing desired structures are segmented. The desired structures being at least the fiducial markers, but potentially also other structures such as hard tissue (bone) and soft tissue structures (nerves or blood vessels),
4. make a surgical planning using software to determine the bone volume which has to be removed,
5. perform a path planning using software to calculate the trajectory or trajectories which should be followed by the surgical tool to remove the volume as defined by step 4,
6. transfer the calculated path/trajectory towards individual joint motions of the robot, using an inverse kinematic algorithm of the robot,
7. prepare the operating area for bone removal,
8. clamp the bone of the skull so that it is rigidly attached to the operating area in six degrees of freedom to the base of the robot, or to an intermediate object, which is then again attached to the base of the robot,
9. use the robot's internal encoders and/or an extra apparatus with sensors that is attached to the base of the robot, to determine the locations of all fiducial markers from step 1 to perform a registration, i.e. coupling of CT data from step 2 onto the physical bone from step 8, and
10. perform the bone removal task with the robot using the encoders of the joints, at least one for every moving axis, using feedback from the encoders and possibly feedback from a force sensor placed between the last revolute joint and the prismatic unit to determine the location of the tip of the surgical instrument with respect to the patient's data obtained in steps 2 and 3 and check this location with respect to the planned trajectory and adjust the trajectory if needed.
